(19) United States
(12) Patent Application Publication
(10) Pub. No.: US 2017/0202436 A1
(43) Pub. Date: Jul. 20, 2017

(54) MINIATURE OBSERVATION MIRROR IMAGING LENS STRUCTURE

(71) Applicant: DEX MEDICAL DEVICE CO., LTD., NEW TAIPEI CITY (TW)

(72) Inventor: CHIEN-LI CHANG, NEW TAIPEI CITY (TW)

(21) Appl. No.: 15/281,063

(22) Filed: Sep. 29, 2016

(30) Foreign Application Priority Data
Jan. 14, 2016 (TW) 105200539

(51) Int. Cl. A61B 1/00; A61B 1/05; A61M 16/04; A61B 1/045; A61B 1/267; G02B 23/24; A61B 1/06

(52) U.S. Cl. CPC A61B 1/00114; G02B 23/2461; G02B 23/2446; G02B 23/243; G02B 23/2423; A61B 1/05; A61B 1/00096; A61B 1/0684; A61B 1/0676; A61B 1/045; A61B 1/267; A61M 16/0488; A61B 1/00018; A61B 1/00045; H04N 5/2256

(57) ABSTRACT

A miniature observation mirror imaging lens structure is disclosed, an outer tube body is in engagement with a front end of a bougie, and only an optical filter, lens and integrated circuit are configured inside the outer tube body in sequence from front to rear, where a light emitting diode (LED) is configured on one side of the optical filter, electronic elements are soldered on the integrated circuit, a lower edge thereof is configured with a plurality of contacts on which electronic elements are soldered; the contacts are connected with conducting lines so as to be in connection with a control circuit configured on the rear end of the bougie, furthermore, the contacts, electronic element and conducting lines are encapsulated with sealing glue, thereby allowing the diameter of the imaging lens to be reduced for facilitating endotracheal intubation operation through the simplification of elements on the front end.

### TECHNICAL FIELD OF THE INVENTION

The present invention relates to a miniature observation mirror imaging lens structure, and more particularly to an endotracheal intubation visual bougie structure adapted to facilitate endotracheal intubation operation by reducing the outer diameter of an imaging lens on the front end of a bougie.

### DESCRIPTION OF THE PRIOR ART

General endotracheal intubation observation mirrors are functioned to allow the observation of a human organ state. A conventional observation mirror, as FIG. 1 shows, is configured with a soft and transparent endotracheal inner tube 1, in which a flexible bougie 2 is inserted, where the front end of the bougie 2 is configured with a tube body 21, inside which elements such as a optical filter, lens and optical element are configured, and on the rear end of which a connector 31 is configured, with the connector 31 being allowed to insert in a control circuit 3. Upon use, the endotracheal inner tube 1 together with the bougie 2 is inserted in a patient's mouth, and the control circuit 3 is connected to a host computer 4 with a display screen 41. Thereafter, the lens is adapted to capture the image of an organ after the optical element is light up, and image information is then transmitted to the connector 31 configured on the rear end of the bougie 2, with the connector 31 being inserted in the control circuit 3, thereby observing the image on the display screen 41 of the host computer 4. However, conventional observation mirrors are limited to the volumes of the elements, causing the imaging lens configured on the front end of the bougie 2 to be unable to be shrunk, the operation of medical staff is relatively inconvenient, and patients feel uncomfortable upon endotracheal intubation.

### SUMMARY OF THE INVENTION

The main object of the present invention is to provide a miniature observation mirror imaging lens structure, allowing the diameter of an imaging lens configured on the front end of a bougie to be reduced substantially for facilitating endotracheal intubation operation, and a patient's discomfort to be alleviated during endotracheal intubation operation.

To achieve the object mentioned above, the present invention proposes a miniature observation mirror imaging lens structure, an outer tube body being in engagement with a front end of a bougie, and only an optical filter, imaging lens, and integrated circuit being configured inside the outer tube body in sequence from front to rear, wherein a light emitting diode (LED) is configured on one side of the optical filter, the imaging lens below the optical filter, and the integrated circuit below the imaging lens, allowing the LED and lens to be respectively connected to the integrated circuit, wherein electronic elements are allowed to be soldered on the integrated circuit, a lower edge of the integrated circuit is configured with a plurality of contacts for the soldering of the electronic elements thereon, and conducting lines are in connection with the contacts so as to be in connection with a control circuit configured on a rear end of the bougie, preventing a printed circuit board from being used, thereby allowing the diameter of the imaging lens configured on the front end of the bougie to be reduced by simplifying elements on the front end.

According to the structure mentioned above, an inner sleeve is configured between the optical filter and lens in advance, thereby positioning the optical filter and lens.

### BRIEF DESCRIPTION OF THE DRAWINGS

FIG. 1 is a perspective view of a visual bougie of a miniature observation mirror;

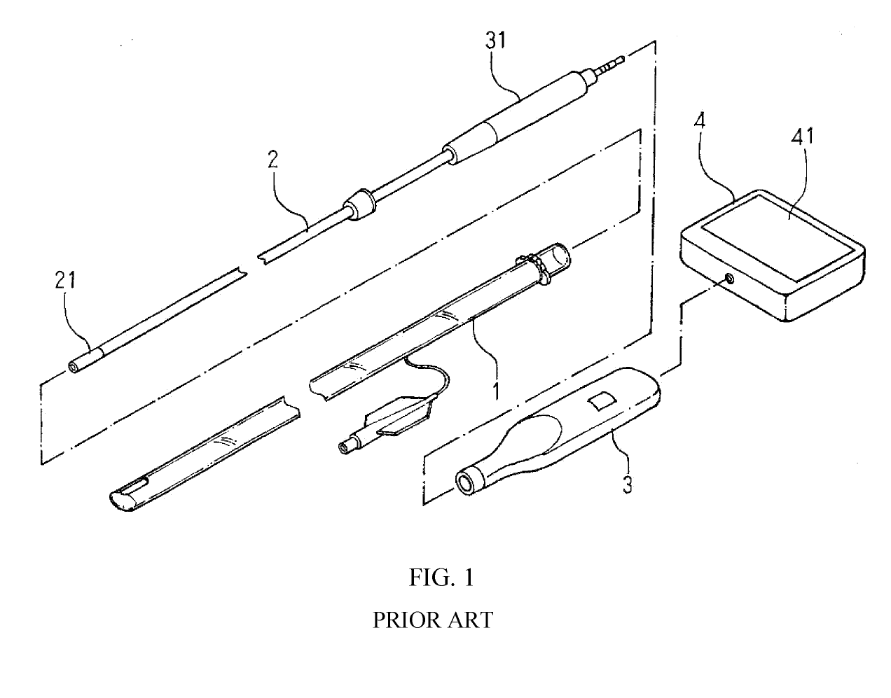

FIG. 2 is a perspective view of the present invention;

FIG. 3 is a cross-sectional view of the present invention;

FIG. 4 is a schematic view of integrated circuit contacts in connection with electronic elements according to the present invention;

FIG. 5 is a schematic view of integrated circuit contacts in connection with a control circuit according to the present invention; and

FIG. 6 is a cross-sectional view of the present invention in an action state.

### DETAILED DESCRIPTION OF THE PREFERRED EMBODIMENTS

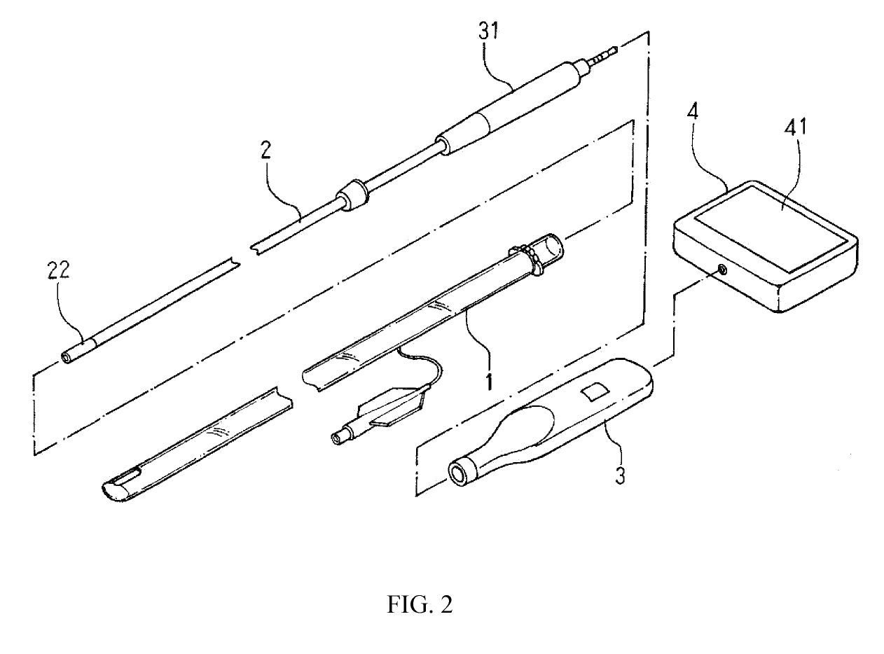

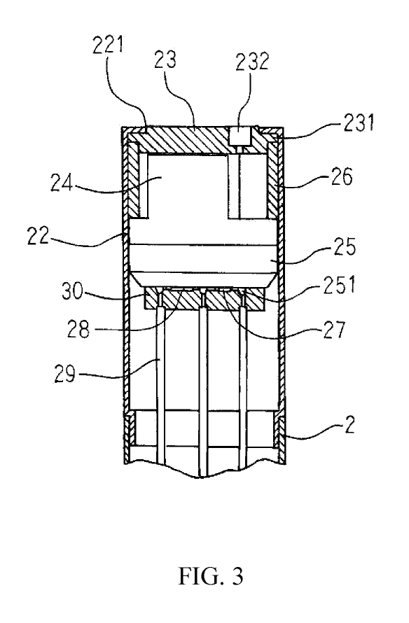

Referring to FIGS. 2 and 3, which respectively are a perspective view and cross-sectional view of the present invention, an observation mirror of the present invention, like conventional ones, is configured with a soft and transparent endotracheal inner tube 1, in which a flexible bougie 2 is inserted.

Furthermore, an outer tube body 22 is engaged with the front end of the bougie 2, and a control circuit 3 is configured on the rear end thereof, where an optical filter 23, imagining lens 24, integrated circuit 25 are configured inside the outer tube body 22 in sequence from front to rear. In addition, an opening 221 is configured on end of the outer tube body 22, and a wall portion 231 of a larger diameter on the optical filter 23, allowing the upper side of the wall portion 231 to be propped against the opening 221 of the outer tube body 22. Furthermore, a light emitting diode (LED) 232 is configured on one side of the optical filter 23, and the imaging lens 24 below the optical filter 23, with an inner sleeve 26 being configured between the optical filter 23 and imaging lens 24, thereby positioning the optical filter 23 and lens 24. Furthermore, the integrated circuit 25 is configured below the imaging lens 24, allowing the LED 232 and lens 24 mentioned above to be respectively connected to the integrated circuit 25.

The circuit board 25 allows electronic elements 27, 28 (e.g. micro resistors, capacitors) to be soldered thereon, the lower edge of which is configured with a plurality of contacts 251, allowing the electronic elements 27, 28 to be soldered on the plurality of contacts and the plurality of contacts 251 to be in connection with conducting lines 29 so as to be in connection with the control circuit 3 configured on the rear end of the bougie 2, where the contacts 251, electronic elements 27, 28 and conducting lines 29 are encapsulated with sealing glue 30, thereby fixing the electronic elements 27, 28 and conducting lines 29 after the electronic elements 27, 28 and conducting lines 29 are soldered on the contacts 251, allowing the outer diameter of the front end of the bougie 2 and further the one of the imaging lens 24 to be reduced for facilitating endotracheal intubation operation.

The miniature observation mirror imaging lens structure is thus formed through the combination of the components mentioned above. Upon use, the bougie 2 is inserted into the endotracheal inner tube 1, the endotracheal inner tube 1 is then inserted into a patient's mouth together with the bougie 2, and the control circuit 3 is connected to a host computer 4 with a display screen 41. Thereafter, the imagining lens 24 captures the images of a organ through the illumination of the LED 232, and image information is then transmitted to the control circuit 3 configured on the rear end of the bougie 2, thereby observing the images on the display screen 41 of the host computer 4. Therefore, the diameter of the outer tube body 22 of the imaging lens 24 configured on the front end of the bougie 2 can be reduced substantially to further reduce the outer diameter of the imaging lens 24 for facilitating the endotracheal intubation operation and reducing the patient's discomfort during the intubation.

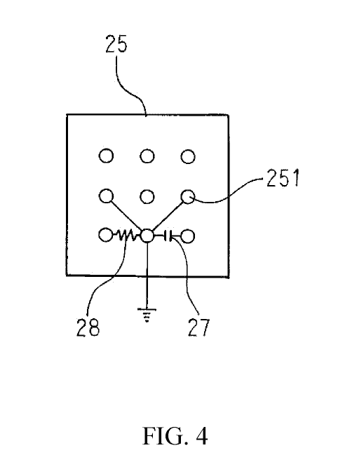

Referring to FIG. 4, which is a schematic view of the contacts of the integrated circuit in connection with the electronic elements according to the present invention, and FIG. 3 again, the integrated circuit 25 of the present invention, in the embodiment, includes the plurality of contacts 251 for the soldering of electronic elements 27, 28 thereon, allowing the electronic elements 27, 28 to be respectively connected across the different contacts 251, thereby preventing a printed circuit board from being used so as to reduce the size of the outer tube body 22 on the front end of the bougie 2, and further the size of the imaging lens 24.

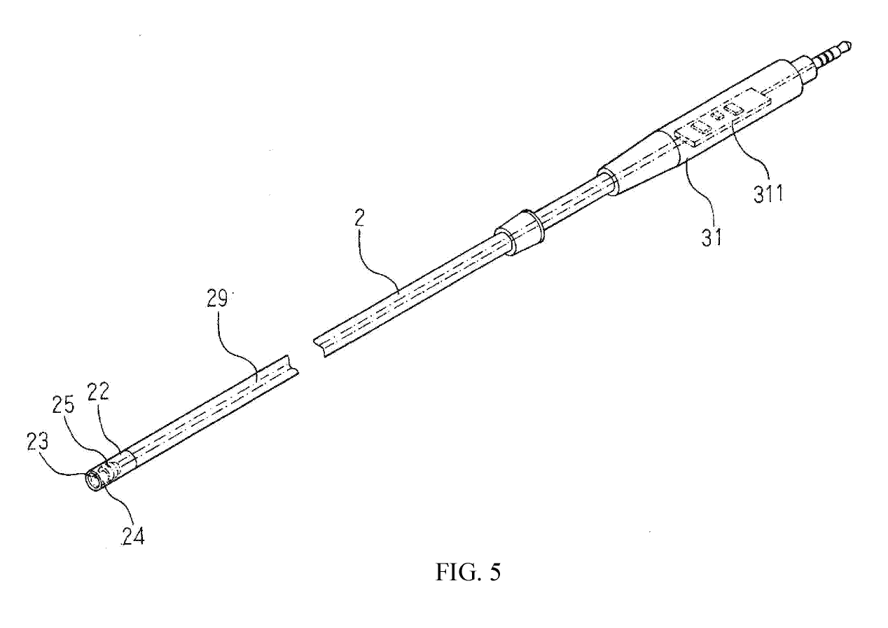

Referring to FIG. 5, which is a perspective view of the integrated circuit in connection with the control circuit according to the present invention, and FIGS. 3 and 4 again, the conducting lines 29 may be connected to a electronic circuit 311 of a connector 31 directly after the electronic elements 27, 28 and conducting lines 29 are soldered on the integrated circuit 25, thereby allowing the elements of the outer tube body 22 on the front end of the bougie 2 to be decreased, the outer diameter of the outer tube body 22 to be reduced, and the outer diameter of the imaging lens 24 to be further shrunk.

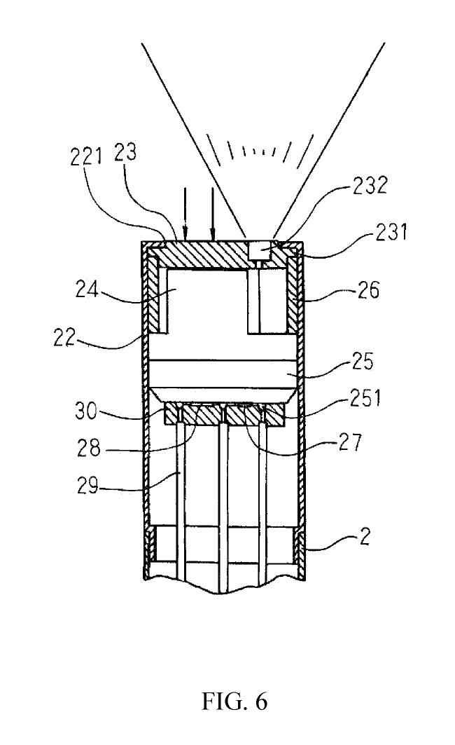

Referring to FIG. 6, which is a schematically cross-sectional view of the present invention in an action state, and FIG. 2 again, the LED 232 configured on the front end of the bougie 2, upon use, is lighted up through the control circuit 3 to illuminate an organ, allowing the lens to capture the images thereof clearly, and image information is then transmitted to the control circuit 3 on the rear end of the endotracheal inner tube 1 through the conducting lines 29 for the processing of the control circuit 3, thereby observing the images on the display screen 41 of the host computer 4.

To sum up, the present invention uses the outer tube body in engagement with the front end of the bougie to coordinate with the optical filter, imaging lens, integrated circuit and electronic elements and conducting lines soldered on the contacts of the integrated circuit configured inside the outer tube body to form a miniature observation mirror imaging lens structure, allowing the outer diameter of the front end of the bougie to be reduced substantially and facilitating the alleviation of a patient's discomfort upon endotracheal intubation operation.

I claim:

1. A miniature observation mirror imaging lens structure, an outer tube body being in engagement with a front end of a bougie, and only an optical filter, imaging lens, and integrated circuit being configured inside said outer tube body in sequence from front to rear, wherein a light emitting diode (LED) is configured on one side of said optical filter, said imaging lens below said optical filter, and said integrated circuit below said imaging lens, allowing said LED and lens to be respectively connected to said integrated circuit,
wherein electronic elements are allowed to be soldered on said integrated circuit, a lower edge of said integrated circuit is configured with a plurality of contacts for the soldering of said electronic elements, and conducting lines are in connection with said contacts so as to be in connection with a control circuit configured on a rear end of said bougie, preventing a printed circuit board from being used, thereby allowing the diameter of said imaging lens configured on said front end of said bougie to be reduced by simplifying elements on said front end.

2. The structure according to claim 1, wherein said contacts, electronic elements and conducting lines are encapsulated with sealing glue to fix said electronic elements and conducting lines after said electronic element and conducting lines are soldered on said integrated circuit.
